(19) United States
(12) Patent Application Publication
(10) Pub. No.: US 2005/0106684 A1
(43) Pub. Date: May 19, 2005

(54) Method and device for carrying out a reaction

(75) Inventors: Jorg Hassmann, Erlangen (DE); Hans Kosak, Bonn (DE); Dirk Kuhlmeier, Nurnberg (DE); Jurgen Krause, Erlangen (DE); Markus Neugebauer, Erlangen (DE); Ugur Ulker, Erlangen (DE)

Correspondence Address: RANKIN, HILL, PORTER & CLARK LLP, 4080 ERIE STREET, WILLOUGHBY, OH, 44094-7836, US

(73) Assignee: NOVEMBER AKTIENGESELLSCHAFT, Erlangen (DE)

(21) Appl. No.: 10/970,931

(22) Filed: Oct. 22, 2004

(30) Foreign Application Priority Data
Oct. 24, 2003 (DE) 103 49 708.0-23

(51) Int. Cl. C12Q 1/68; C12P 19/34

(52) U.S. Cl. 435/91.2; 435/6; 423/659

(57) ABSTRACT

The invention relates to a method for carrying out a reaction in an unmoved liquid in a capillary which is open at at least one end, with gas bubbles forming in the liquid, with a detergent being added to the liquid for the purpose of changing at least one property of the gas bubbles which are being formed and with the capillary being arranged, or having been arranged, at at least an angle to the horizontal which is such that the gas bubbles in the capillary ascend to a surface of the liquid.

The invention relates to a method for carrying out a reaction in a capillary, to a use and to a device for carrying out the method.

WO 01/2.3093 discloses a method for carrying out an amplification reaction. In this method, a sample which contains a target nucleic acid sequence, primers, nucleotides, enzymes for the amplification reaction, a buffer system and, where appropriate, a detergent is provided in a recess in a device. This sample is then used to carry out a PCR reaction. The device comprises a thermally conducting layer and a covering layer having at least one recess of up to 1000 μm in depth. The device is sealed in a pressure-tight manner before the reaction is carried out. Without the device being sealed, heating to the temperatures which are customary in PCR would lead to the formation of gas bubbles. With the proposed configuration of the device, the resulting gas bubbles would at least partially expel the reaction liquid from the device.

A disadvantage of the method is that it is not carried out with an open device. When samples are processed in an automated manner, it is disadvantageous if the reaction vessel has to be sealed in a pressure-tight manner on each occasion. Faults can arise in connection with the pressure-tight sealing. Furthermore, pressure-tight sealing in automated methods where a high sample throughput is of importance is an additional operational step, which demands time and instrumental input, aside from the opening of the reaction vessel which is subsequently required. Another disadvantage of the method is that, because of the necessity for resistance to pressure, high demands have to be placed on the material of the device, with these demands increasing the cost of the device.

EP 0 636 413 B1 discloses a method for nucleic acid amplification which is carried out in at least one open capillary. In this method, a reaction mixture is pumped through regions of the capillary which are at different temperatures or a heat source is moved along the capillary. Bubble formation can result in a part of the reaction mixture in the capillary being separated from another part. The problem is solved by the reaction mixture being moved in a step-like manner by means of a stepping motor, with this movement allowing the part of the reaction mixture which is separated off by a bubble to catch up with the main part of the reaction mixture. Furthermore, the problem can be solved by the part of the capillary whose temperature is otherwise not controlled being kept at an elevated temperature. In addition, it is proposed, for solving the problem, that an internal surface roughness of the capillary be reduced and/or its inner wall be coated with a polymer such as parylene. As another alternative, it is proposed that a denaturing agent be taken up into the reaction mixture. This agent can be formamide, glycerol or acetamide. This thereby lowers the denaturation temperature and annealing/extension temperature by in each case about 5° C. The PCR can consequently be carried out at temperatures at which the formation of gas bubbles is reduced.

Another problem which is associated with gas bubbles which form in a liquid in an open capillary is that growing gas bubbles which appear on the wall of the capillary can displace the liquid in the capillary. As a result, the liquid can be expelled from the capillary or pushed into a region of the capillary in which a desired reaction condition, such as a particular temperature, does not exist.

The object of the present invention is to provide a method which prevents the problem of liquid being displaced by gas bubble formation in reactions which are carried out in open capillaries. In addition, the intention is to specify a device which is suitable for carrying out the method, as well as a use.

This object is achieved by means of the features of claims 1, 40 and 72. Expedient embodiments ensue from the features of claims 2 to 39, 41 to 71 and 73 to 78.

The invention provides a method for carrying out a reaction in an unmoved liquid in a capillary which is open at at least one end, with gas bubbles being formed in the liquid. A detergent is added to the liquid in order to alter at least one property of the gas bubbles which are being formed and the capillary is arranged, or is already arranged, in at least an angle to the horizontal which is such that the gas bubbles in the capillary ascend to a surface of the liquid. A liquid is unmoved within the meaning of the invention if it is not moved as a whole in the capillary. A movement as a whole would, for example, be a back and/or forth movement or a transportation of the liquid through a pump. However, this does not exclude movements within the liquid, for example as a result of convection or as a result of the ascent of gas bubbles. The surface of the liquid is to be understood as being the most highly located boundary surface of the liquid in the capillary. A capillary is understood here as being a thin tube whose internal width is such that it can be filled by a gas bubble which forms in the liquid in the absence of detergent. While, in this present case, a capillary can be a tube which has such a small internal diameter that it becomes partially filled with liquid as a result of capillary forces, it can also be a tube having a large internal diameter. “Internal width” is understood here, and in that which follows, as being the largest distance from one inner side of a wall to the other inner side of a wall in cross section. In the case of a circular cross section, this is the internal diameter. The property which is altered by the detergent can be the size of the gas bubbles which are forming, the adherence of the gas bubbles to the wall of the capillaries or the dimensional stability of the gas bubbles.

The effect of adding the detergent is to cause the gas bubbles to usually become detached at a smaller size than without detergent, and to ascend to the surface of the liquid, where they burst. Consequently, the gas bubbles frequently do not reach a size which spans the interior of the capillary such that a part of the liquid contained in the capillary is thereby separated from another part. However, even if a gas bubble should reach a size which spans the interior of the capillary, it does not, in the presence of detergent, expel any liquid from the capillary when it ascends in the capillary. In the presence of detergent, the gas bubble is so flexible or readily deformable that, when the gas bubble ascends in the capillary, the liquid can flow past the gas bubble. Furthermore, the detergent has the effect of causing the gas bubbles to have less tendency to adhere to the wall of the capillary. The detergent thereby promotes the ascent of the gas bubbles to the surface of the liquid. Adding the detergent furthermore has the affect of causing gas bubbles which are being formed to detach more readily, even from hydrophobic vessel surfaces, such as plastic surfaces, and thus frequently also at a smaller size than without detergent. In the method according to the invention, the gas bubbles do not interfere with automated sample processing.

A source of the gas bubbles can be air which is dissolved in the liquid. The problem of gas bubble formation can usually be reduced by degassing the liquid in a vacuum before carrying out the reaction. An advantage of the method according to the invention is that such degassing is not required. Another advantage is that it is not necessary, for the purpose of avoiding gas bubbles, to enclose the liquid in the capillary in a pressure-tight manner. The method according to the invention thereby substantially reduces the input of apparatus in connection with the automated sample processing. As a result of the capillary not having to withstand any elevated pressure, it can be designed to be very thin-walled using simple materials, and consequently inexpensively, in particular as a component of a disposable unit which is to be used for the sample processing in an automated appliance. The fact that the capillary is thin-walled advantageously also makes it possible for heat to be readily and rapidly transferred from the exterior into the capillary. A disposable unit is understood as being a device which is suitable, and intended, for implementing the method once and, after that, is usually not used any further.

The gas bubbles can be formed by a gas-forming reaction, by boiling or by gassing out a gas which is dissolved in the liquid. They are formed, in particular, when the liquid is heated to a temperature. This temperature can also be below the boiling point of the liquid. The heating of the liquid to a temperature can be effected by means of supplying heat from the exterior or by means of the reaction being exothermic. The liquid can also be repeatedly heated to the temperature in connection with the reaction, for example a polymerase chain reaction (PCR). Preferably, the reaction is carried out under constant pressure, in particular atmospheric pressure. Carrying out the reaction under atmospheric pressure substantially reduces the input of apparatus, particularly in connection with processing the liquid in an automated manner.

The angle to the horizontal can be between 10° and 90°, in particular between ₄₅° and 90°, preferably between 80° and 90”, preferably 90°. At an angle of 90°, the gas bubbles which are being formed can ascend vertically in the capillary. The greater the angle of the capillary is to the horizontal, the easier it is for the gas bubbles to become detached from the wall and ascend to the surface. The easier it is for the gas bubbles to become detached from the wall, the smaller is their size on becoming detached.

The detergent can be a nonionic detergent, in particular Tween 20, Nonidet P40 or Triton X-100. The amount of detergent added to the liquid can be such that the concentration of the detergent in the liquid is from 0.01 to 5% (w/v), preferably from 0.02 to 5% (w/v), most preferably from 0.05 to 5% (w/v), in particular from 0.1 to 5% (w/v) or from 0.5 to 5% (w/v). Astonishingly, it has been found that this detergent concentration is enough to ensure interference-free ascension of the gas bubbles. Astonishingly, 5% (w/v) detergent in the liquid does not interfere with many reactions, such as a polymerase chain reaction. The detergent can be added to the liquid prior to the reaction, in particular together with reagents for implementing the reaction. In particular, the reagents for implementing the reaction and/or the detergent can be placed in the capillary initially, preferably in dry form. They are then added to the liquid by the liquid being introduced into the capillary. The liquid can be introduced into the capillary by being sucked into it by means of capillary forces or by means of applying a negative pressure or can be pushed into it by means of a positive pressure.

The reaction is preferably a nucleic acid amplification reaction, in particular a polymerase chain reaction (PCR). In this connection, it is necessary to repeatedly heat the liquid to a temperature at which gas bubbles can form. In this connection, the temperature can be between 80° C. and 100° C., in particular between 85° C. and 97° C., preferably 95° C. The capillary can consist of glass. However, it preferably consists of a plastic which is, in particular, hydrophobic and preferably rigid. Hydrophobic plastics make it possible to produce a device which is suitable for implementing the reaction, in particular a PCR, as an inexpensive disposable article.

The plastic is preferably a plastic which does not inhibit the PCR, preferably polycarbonate, polypropylene or polyethylene. The plastic is to be selected such that it is thermally stable at the temperatures which arise in connection with the PCRI i.e. such that it is not significantly deformed at these temperatures. Whether a plastic is a plastic which does not inhibit the PCR has to be checked for the given plastic or the given plastic type in each individual case. Even in the case of plastics which apparently consist of the same material, one batch of the plastic can inhibit the PCR while another batch is compatible with the PCR. The reason for this is unclear.

It is advantageous if the capillary is shaped such that, during their ascent to the surface of the liquid, the gas bubbles are not retained at a projection, at a local high point or at a site in the capillary which does not have an angle to the horizontal which is sufficient to allow the gas bubbles to further ascend, from that site, to the surface of the liquid. In particular, the capillary can have a linear shape. The internal width of the capillary can be at least 0.2 mm, preferably at least 0.3 mm, and at most 3 mm, in particular at most 2 mm. These measurements have proved to be particularly advantageous, in particular for implementing a PCR in which heat is supplied from the exterior to the liquid in the interior of the capillary. The cross sectional area of the capillary can be maximally 10 mm², in particular maximally 4 mm², preferably maximally 2 mm². A length of the capillary of from 10 to 100 mm, in particular from 20 to 30 mm, preferably 25 mm, has proven to be advantageous. The abovementioned measurements are particularly advantageous for carrying out a PCR in the capillary. The volume of the liquid can be from 0.5 to 500 μl, in particular from 5 to 50 μl, preferably from 10 to 30 μl.

Prior to implementing the reaction, the capillary can be formed by an open longitudinal side of an elongated channel-shaped recess in a substrate, being sealed with a plastic film which is, in particular, self-adhering. This enables the capillary to be produced in a simple manner. Furthermore, particularly as a result of the fact that it can be designed to be very thin, the plastic film permits good heat transfer from a heating element into the interior of the capillary. The plastic film is preferably from 0.01 to 0.5 mm, in particular from 0.2 to 0.5 mm, preferably from 0.3 to 0.4 mm, in thickness. The plastic film can form at least ⅕, preferably at least ¼, in particular at least ⅓, of the area of the longitudinal wall of the capillary. The plastic film can consist of a soft and flexible material or else of a hard and nonflexible material and, in particular, of the same material as the substrate. The substrate is a solid basic substance, which preferably consists of the plastic. The plastic film can be bonded to the substrate using an adhesive, in particular a hot-melt adhesive, preferably by means of laminating. This enables the capillary to be produced particularly simply and inexpensively. In the laminating, the plastic film is laid on the substrate. Either the plastic film or the substrate is coated with a hot-melt adhesive. Sufficient heat is then supplied externally to melt the hot-melt adhesive. At the same time, the plastic film and the substrate are pressed together. After the subsequent cooling, the substrate and the plastic film are bonded firmly to each other. The plastic film can also be bonded to the substrate by means of melting, in particular thermal melting. For this, the plastic film and/or the substrate can be joined together in a state in which it/they is/are still hot and fusible from having been produced or after the plastic film and/or the substrate has/have been melted by heating. The thickness of the plastic film can also be reduced by rolling it, in the hot state, on the substrate while exerting pressure.

The capillary preferably has a first aperture and a second aperture, which apertures are arranged, in particular, at opposing ends of the capillary. The capillary can be filled with liquid by way of the first aperture and emptied by way of the first aperture or the second aperture. The filling and/or emptying can be effected using pressure or negative pressure. In this connection, the first aperture is preferably arranged below the surface of the liquid and the second aperture is arranged above the surface of the liquid. The second aperture can be used for equalizing pressure with the environment. The first aperture can be sealed for implementing the reaction. The second aperture can have an internal width which is smaller than the internal width of the capillary. This can thereby reduce any escape of vaporized liquid. The liquid can condense in the region around the second aperture. The internal width of the second aperture can be at most 0.3 mm, preferably at most 0.2 mm. An appliance for the automated processing of samples can be connected to the first aperture and/or the second aperture, in particular by way of liquid-conducting channels which are contained in the device. The filling and emptying of the capillary can then take place in an automated manner in exactly the same way as the heating or cooling of the capillary. The liquid is preferably heated by heat being supplied to it through the plastic film from a heating element or by heat being supplied to it by a heating element which is arranged within the capillary, within a wall of the capillary or within the plastic film. The liquid can also be heated or cooled by a gas or air stream of the appropriate temperature being blown onto the capillary.

It is particularly advantageous if the capillary is only partly, in particular only up to a maximum of 80% of its volume, filled with the liquid. Heat can then be supplied to the capillary only in a region of the capillary which is filled with liquid such that the liquid which is vaporized in the reaction can condense in a residual region of the capillary, in particular a region which is arranged below the second aperture, and can flow back into the liquid which remains. The condensing can be supported by the residual region of the capillary being cooled, in particular by means of a Peltier element or an enlarged surface which radiates off heat.

The method is preferably used for the automated sample working-up, synthesis and/or analysis of biopolymers. The capillary can be a component of a disposable unit which is, in particular, inserted into an appliance for the automated processing of samples. In this connection, the automated processing appliance can be responsible, in particular, for filling and emptying the capillary with the liquid, supplying and subsequently transporting the liquid and controlling the temperature of the capillary.

It is advantageous if an outflow of heat from the capillary into parts of the substrate is reduced by a means for interrupting the heat transfer, in particular a recess, which runs essentially parallel to the capillary, being arranged in the substrate between the capillary and the parts of the substrate. Interrupting the heat transfer in this way reduces the thermal inertia of the capillary and prevents or reduces an unwanted heating of other components which are arranged in the parts of the substrate.

The invention furthermore relates to a device for carrying out a method according to the invention, which device contains at least one elongated reaction chamber which has at least one first aperture, with at least one part of the longitudinal wall of the reaction chamber being formed by a plastic film. The reaction chamber is formed from a capillary having a round or angular cross section. Advantageous dimensions of the capillary can be obtained from the above statements with regard to the method according to the invention. The reaction chamber is formed in a substrate by at least one elongated, channel-shaped recess which is entirely or partially sealed with the plastic film. A means for interrupting the transfer of heat from the capillary into parts of the substrate, in particular a recess which runs essentially parallel to the capillary, is provided in the substrate, between the capillary and the parts of the substrate.

The plastic film makes it possible, in particular, to produce, in a very simple manner, a device having a multiplicity of reaction chambers which are arranged in parallel and in which a shared plastic film in each case forms a part of the reaction chamber. Furthermore, the plastic film, particularly when it is thin, permits very good heat transfer. Since, in the method according to the invention, the plastic film does not have to withstand any positive pressure, it can be made to be very thin. Preferably, its thickness is from 0.01 to 0.5 mm, in particular from 0.2 to 0.5 mm, preferably from 0.3 to 0.4 mm. The plastic film can consist of a soft and flexible material or else of a hard and nonflexible material, in particular of the same material as the substrate.

The substrate can consist of a plastic which is, in particular, hydrophobic and preferably rigid. For implementing a PCR, it is advantageous if the plastic is a plastic which does not inhibit the PCR, preferably polycarbonate, polypropylene or polyethylene. The substrate can be an injection-molded part, in exactly the same way as the plastic film. The recess can be formed in the substrate during the injection molding or, after that, by means of stamping, punching, milling or etching. The plastic film is preferably self-adhering. In one embodiment according to the invention, the plastic film is bonded to the substrate by means of melting, in particular thermal melting, or using an adhesive, in particular a hot-melt adhesive, preferably by means of laminating. The plastic film preferably forms at least ⅕, preferably at least ¼, in particular at least ⅓, of the area of the longitudinal wall of the capillary. The volume of the reaction chamber can be from 0.5 to 500 μl, in particular 5-50 μl, preferably from 10 to 30 μl. The reaction chamber can have a first aperture and a second aperture, which apertures are preferably arranged at opposing ends of the reaction chamber. The first aperture and/or second aperture is/are preferably arranged in the substrate, i.e. not in the plastic film.

The first aperture is preferably arranged in a part of the reaction chamber which is envisaged for taking up liquid and the second aperture is arranged in the other part of the reaction chamber. The internal width of the second aperture is preferably smaller than the internal width of the capillary. In particular, the internal width of the second aperture can be at most 0.3 mm, preferably at most 0.2 mm. Furthermore, the device can contain a heating element, in particular within the reaction chamber, within a wall of the reaction chamber or within the plastic film. This element can be used to supply heat to the interior of the reaction chamber. It is particularly advantageous to supply heat to the interior of the reaction chamber by way of the plastic film.

It is advantageous if the heating element is arranged such that heat can only thereby be supplied to a region of the reaction chamber which is envisaged for taking up liquid, so that vaporized liquid can condense in the other region of the reaction chamber. A cooling element, in particular a Peltier element or an enlarged surface for radiating off heat, can be provided at the other region of the reaction chamber. The reaction chamber is preferably shaped such that, when the reaction chamber is at a sufficient angle to the horizontal, gas bubbles which are formed in the liquid therein can ascend to the surface of the liquid without being retained, in this connection, at a projection, at a local high point or at a site in the reaction chamber which does not have a sufficient angle to the horizontal. For this purpose, the reaction chamber preferably has an angle to the horizontal of between 10° and 90°, in particular of between 45° and 90°, preferably of between 80° and 90°, preferably 90°. Advantageously, the reaction chamber has a linear shape.

The method which is to be carried out using the device is preferably a nucleic acid amplification reaction, in particular a polymerase chain reaction. The reaction chamber, the plastic film and the adhesive, which is present, where appropriate, are advantageously selected such that they withstand a temperature of between 80° C. and 100° C., in particular of between 85° C. and 97° C., preferably 95° C. Withstanding means that no significant deformation, softening or, in the case of the adhesive, a reduction in its adhesive force, occurs at this temperature.

Advantageously, the reaction chamber is connected, at the first and/or the second aperture, in particular by way of liquid-conducting channels which are contained in the device, to an appliance for the automated processing of samples, or can be connected to the appliance, in particular by insertion into the appliance. The appliance can, for example, be designed such that it activates pistons which are contained in the device in order, in this way, to convey liquid into or out of the reaction chamber.

The device can possess several reaction chambers which are, in particular, arranged parallel to each other. It is particularly advantageous if the plastic film extends over several of the reaction chambers and in each case forms a part of the longitudinal wall of each of these reaction chambers. In this way, it is possible to provide many reaction chambers in one operational step. Advantageously the device contains, in at least one of the reaction chambers, reagents for carrying out the method, in particular reagents for implementing a PCR and/or a detergent, preferably in dry form.

The invention furthermore relates to the use of a detergent for changing a property of gas bubbles which are being formed in connection with a reaction in an unmoved liquid in a capillary which is open at at least one end, with the capillary having at least an angle to the horizontal which is such that the gas bubbles in the capillary ascend to a surface of the liquid. The detergent can be a nonionic detergent, in particular Tween 20, Nonidet P40 or Triton X-100. The detergent can be used in the concentrations which have already been mentioned. The reaction can be part of an automated sample working-up, synthesis and/or analysis of biopolymers. The capillary can be a component of a disposable unit which has been inserted, in particular, into an appliance for automated processing. The detergent is preferably used in a method according to the invention.

It is furthermore advantageous if the capillary is provided by a device according to the invention.

Advantageous embodiments of the invention are explained below with the aid of an implementation example.

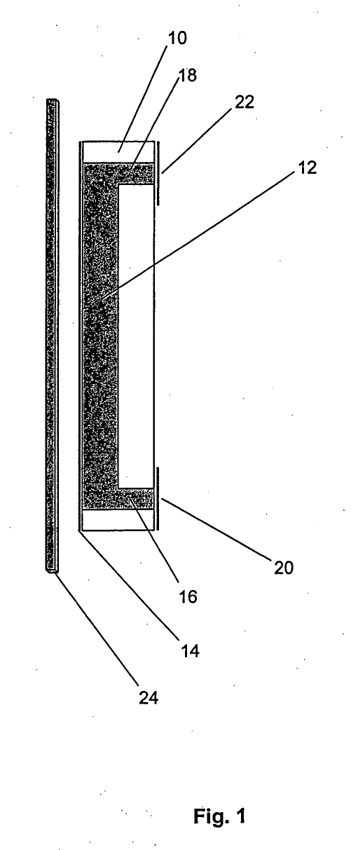

FIG. 1 shows a diagram of the construction of a device according to the invention possessing a heating element,

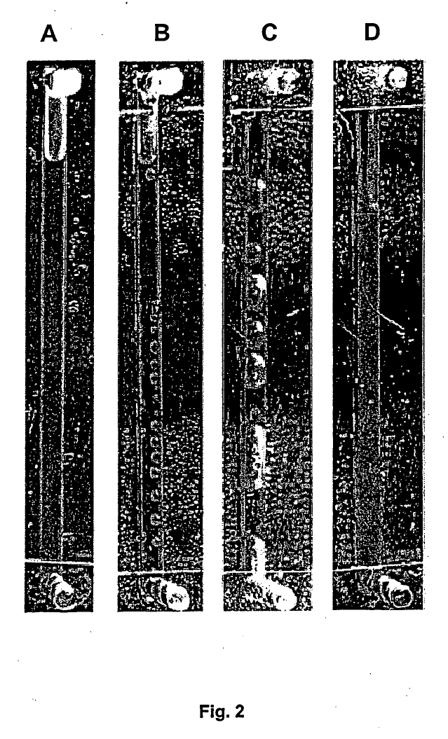

FIG. 2 shows photographs of a device according to the invention before and after heating aqueous liquids contained in it,

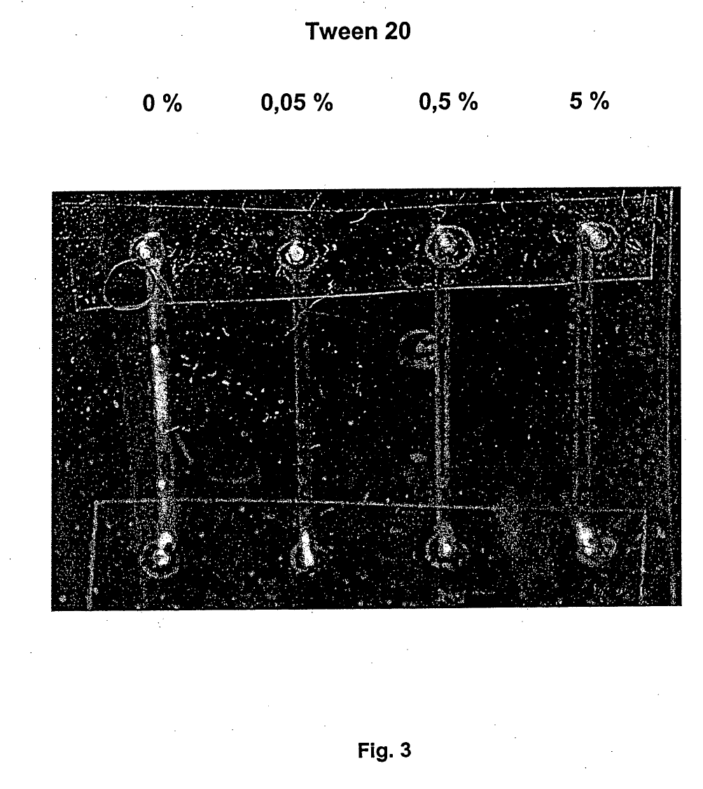

FIG. 3 shows a photograph of a device according to the invention in connection with carrying out a method according to the invention using different detergent concentrations,

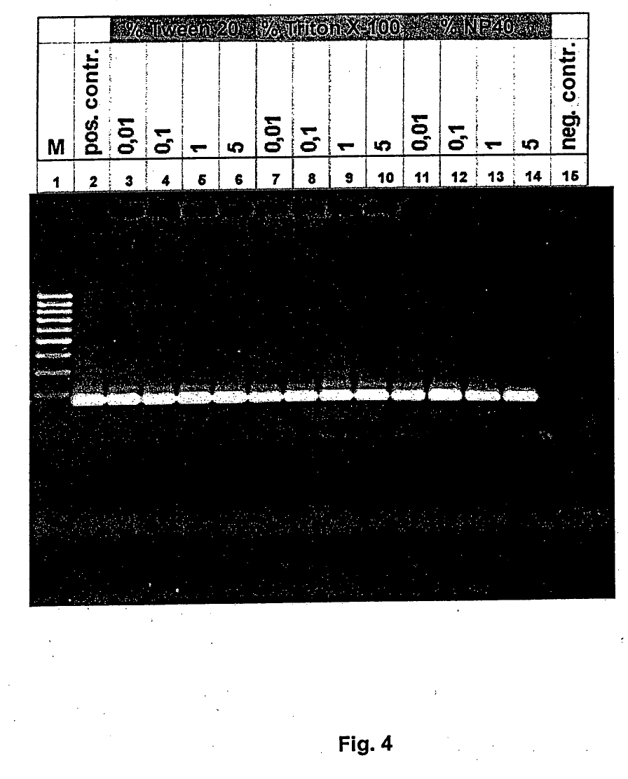

FIG. 4 shows a separation, by gel electrophoresis, of PCR products which have been obtained after carrying out a conventional PCR in PCR reaction vessels in the presence of various detergents of differing concentrations,

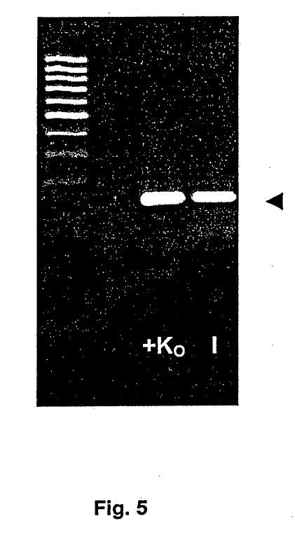

FIG. 5 shows a separation, by gel electrophoresis, of products of the amplification of a first gene by means of PCR, and

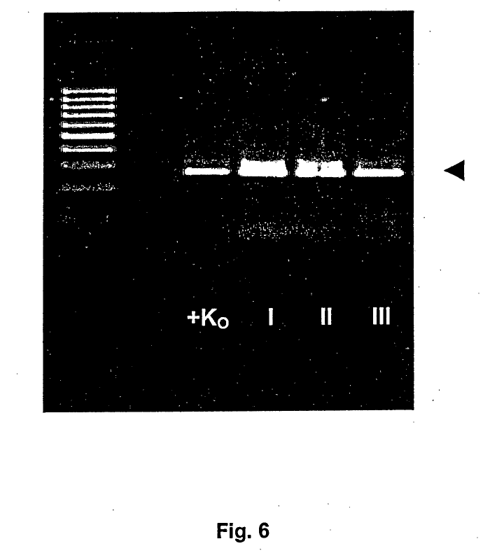

FIG. 6 shows a separation, by gel electrophoresis, of products of the amplification of a second gene by means of PCR.

A linear reaction chamber 12 was milled out, as a recess measuring 1.0 mm×1.3 mm×25 mm, in a polycarbonate substrate 10 and sealed with a self-adhering plastic film 14. The plastic film can, for example, be “Polyester Film Tape 850 Transparent” from 3M, Industrial Tape and Specialties Division, 3M Center, Building 220-7W-03, St. Paul, Minn. 55144-1000, USA. A first aperture 16 and a second aperture 18 to the reaction chamber 12, with each aperture having a diameter of 1 mm, were drilled in the substrate. After the reaction chamber 12 had been filled to ⅔ of its volume with a test solution, the first aperture 16 and the second aperture 18 were in each case sealed off with another self-adhering plastic film 20, 22. A hole of 0.3 mm in diameter was pierced through the plastic film 22 sealing the second aperture 18 in order to ensure equalization of pressure during the reaction. The temperature of the reaction chamber 12 was controlled, through the self-adhering plastic film 14, by means of a vertically aligned heating element 24 belonging to an in-situ thermocycler of the “Cyclone Gradient” type from PEQLAB Biotechnologie GmbH, Carl-Thiersch-Str. 2b, D-91052 Erlangen, Germany.

In order to simulate a PCR, the device according to the invention was initially filled with a 150 mmol/l solution of NaCl in H₂O which contained 0.03% (w/v) of the dye amaranth for improved visualization. The temperature of this solution was controlled by means of a heating element belonging to an in-situ thermocycler. The following temperature cycle was passed through:

| | |
|---|---|
| Step 1: | 5 min at 95° C. |
| Step 2: | 1 min at 95° C. |
| Step 3: | 1 min at 55° C. |
| Step 4: | 1 min at 72° C. |
| Step 5: | 2 min at 72° C. |

The steps numbered 2 to 4 were repeated 35 times.

FIG. 2 shows the device before setting a temperature in A, after the temperature was first set to 95° C. in B and after several temperature cycles had been passed through in C. This shows that bubble formation has an interfering effect as the number of temperature cycles increases and pushes the liquid out of the device. In D, FIG. 2 shows the device after having passed through the same number of temperature cycles as in C but with 5% (w/v) of the nonionic detergent Triton X-100 additionally being present in the solution. In this case, the gas bubbles which formed ascended to the surface of the liquid while being of smaller size. They did not remain attached to the wall of the reaction chamber and did not displace the solution contained in the reaction chamber.

FIG. 3 shows four reaction chambers which are arranged in parallel in a polycarbonate substrate. They are filled with a solution of 0.03% (w/v) amaranth, 150 mmol of NaCl/1 in H₂O, with, in addition, the concentrations given in percent (w/v) of the nonionic detergent Tween 20 being present in each case. The figure shows the reaction chambers after having passed through 35 of the above-described temperature cycles. As a result of their size, and as a result of their remaining in the reaction chamber without ascending, the bubbles which are formed in the solution without detergent displace the dyed solution out of the reaction chamber after a few cycles. By contrast, concentrations of 0.05, 0.5 and 5% (w/v) Tween 20 proved to be equally efficient in ensuring reliable ascension of the small gas bubbles which are formed such that no solution is displaced from the reaction chambers in these cases.

The polymerase chain reactions described below were carried out in the following reaction mixtures in a volume of 30 μl in each case:
200 μmol of each dNTP/1
0.33 μmol of primer 1 (sense)/1
0.33 μmol of primer 2 (antisense)/1
1.5 U of Taq DNA polymerase (“SAWADY Taq DNA polymerase” from PEQLAB Biotechnologie GmbH)
1×PCR buffer Y (“reaction buffer Y” from PEQLAB Biotechnologie GmbH)
17 ng of human genomic DNA
Detergent: as indicated in each case

The following temperature cycle was passed through:

| | |
|---|---|
| Step 1: | 5 min at 95° C. |
| Step 2: | 1 min at 95° C. |
| Step 3: | 1 min at xx° C. |
| Step 4: | 1 min at 72° C. |
| Step 5: | 2 min at 72° C. |

xx° C. denotes the annealing temperature which is in each case indicated. The steps numbered 2 to 4 were repeated 35 times.

After the PCR, the reaction solutions were subjected to electrophoresis in 2% (w/v) agarose gels and DNA which was present was visualized by ethidium bromide staining and UV light irradiation.

In order to examine the question of whether a PCR can also be carried out in the presence of various concentrations of different detergents, a 169 base pair fragment of human genomic DNA encoding factor II (prothrombin) was amplified in the presence of these detergents. Oligonucleotides having the sequences SEQ ID NO 1 and SEQ ID NO 2 in accordance with the enclosed sequence listing were used as primers. The annealing temperature was 55° C. The PCR was carried out without detergent as a positive control (“pos. Contr.”) while it was carried out without the DNA to be amplified as a negative control (“neg. Contr.”). The result is shown in FIG. 4. It can be seen from this that adding the representatively selected detergents Tween 20, Triton X-100 and Nonidet P40 (NP 40) up to a concentration of 5% (w/v) did not have any inhibitory effect on the specific amplification when compared with the positive control.

A fragment of the gene encoding human TNFCC (tumor necrosis factor alpha) was amplified in another PCR. Oligonucleotides having sequences SEQ ID NO 3 and SEQ ID NO 4, as shown in the enclosed sequence listing, were used as primers for this PCR. Reaction I was carried out, at an annealing temperature of 55° C., in a capillary of a device according to the invention. The angle of the capillary to the horizontal was 90°. As a control, +K₀, the same reaction was carried out in a conventional PCR reaction vessel. The reaction mixtures, as separated by gel electrophoresis after the PCR had been carried out, are shown in FIG. 5. In this experiment, a specifically amplified 131 base pair DNA fragment was found both in the case of +K₀ and in the case of I.

FIG. 6 shows the result of PCR reactions I, II and III, which were carried out in three parallel reaction chambers in the device according to the invention, and of a reaction +K₀ which was carried out, for control, in a PCR reaction vessel. The annealing temperature was in each case 58° C. The angle of the reaction chamber to the horizontal was 90°. The experiment involved amplifying a fragment of the gene encoding human factor V (proaccelerin). Oligonucleotides having the sequences SEQ ID NO 5 and SEQ ID NO 6 in accordance with the enclosed sequence listing were used as primers for this purpose. Lanes +K₀, I, II and III in each case show a specific reaction product of 267 base pairs in length.

The results shown in FIGS. 5 and 6 demonstrate that the polymerase chain reactions can be carried out in the devices according to the invention just as efficiently and specifically as in conventional PCR reaction vessels.

### Reference Number List

10 Substrate
12 Reaction chamber
14 Plastic film
16 First aperture
18 Second aperture
20 Self-adhering plastic film
22 Further self-adhering plastic film
24 Heating element

1. A method for carrying out a reaction in an unmoved liquid in a capillary which is open at at least one end, with gas bubbles being formed in the liquid, which comprises adding a detergent to the liquid in order to alter at least one property of the gas bubbles which are being formed and the capillary being arranged, or already having been arranged, in at least an angle to the horizontal which is such that the gas bubbles in the capillary ascend to a surface of the liquid.

2. The method as claimed in claim 1, wherein the gas bubbles form when the liquid is heated to a temperature, in particular up to below the boiling point of the liquid.

3. The method as claimed in claim 2, wherein the liquid is repeatedly heated to the temperature in connection with the reaction.

4. The method as claimed in one of the preceding claims, wherein the reaction is carried out under constant pressure, in particular atmospheric pressure.

5. The method as claimed in one of the preceding claims, wherein the angle is between 10° and 90°, in particular between 45° and 90°, preferably between 80° and 90°, preferably 90°.

6. The method as claimed in one of the preceding claims, wherein the detergent is a nonionic detergent, in particular Tween 20, Nonidet P40 or Triton X-100.

7. The method as claimed in one of the preceding claims, wherein sufficient detergent is added to the liquid for the concentration of the detergent in the liquid to be from 0.01 to 5% (w/v), preferably from 0.02 to 5% (w/v), most preferably from 0.05 to 5% (w/v), in particular.from 0.1 to 5% (w/v) or from 0.5 to 5% (w/v).

8. The method as claimed in one of the preceding claims, wherein the detergent is added to the liquid prior to the reaction, in particular together with reagents for implementing the reaction.

9. The method as claimed in one of the preceding claims, wherein reagents for implementing the reaction and/or the detergent are placed in the capillary initially, particularly in dry form.

10. The method as claimed in one of the preceding claims, wherein the reaction is a nucleic acid amplification reaction, in particular a polymerase chain reaction (PCR).

11. The method as claimed in one of the preceding claims, wherein the temperature is between 80° C. and 100° C., in particular between 85° C. and 97°C., preferably 95° C.

12. The method as claimed in one of the preceding claims, wherein the capillary consists of a plastic which is, in particular, hydrophobic and preferably rigid.

13. The method as claimed in claim 12, wherein the plastic is a plastic which does not inhibit the PCR, preferably polycarbonate, polypropylene or polyethylene.

14. The method as claimed in one of the preceding claims, wherein the capillary is shaped such that, during their ascent to the surface of the liquid, the gas bubbles are not retained at a projection, at a local high point or at a site in the capillary which does not have an angle to the horizontal which is sufficient to allow the gas bubbles to further ascend, from that site, to the surface of the liquid.

15. The method as claimed in one of the preceding claims, wherein the capillary has a linear shape.

16. The method as claimed in one of the preceding claims, wherein the internal width of the capillary is at least 0.2 mm, preferably at least 0.3 mm.

17. The method as claimed in one of the preceding claims, wherein the internal width of the capillary is at most 3 mm, in particular at most 2 mm.

18. The method as claimed in one of the preceding claims, wherein the cross sectional area of the capillary is maximally 10 mm², in particular maximally 4 mm², preferably maximally 2 mm²

19. The method as claimed in one of the preceding claims, wherein the length of the capillary is from 10 to 100 mm, in particular from 20 to 30 mm, preferably 25 mm.

20. The method as claimed in one of the preceding claims, wherein the volume of the liquid is from 0.5 to 500 μl, in particular from 5 to 50 μl, preferably from 10 to 30 μl.

21. The method as claimed in one of the preceding claims, wherein, prior to implementing the reaction, the capillary is formed by an open longitudinal side of an elongated channel-shaped recess in a substrate (10), which preferably consists of the plastic, being sealed with a plastic film (14) which is, in particular, self-adhering.

22. The method as claimed in claim 21, wherein the plastic film (14) forms at least ⅕, preferably at least ¼, in particular at least ⅓, of the area of the longitudinal wall of the capillary.

23. The method as claimed in claim 21 or 22, wherein the plastic film (14) is bonded to the substrate (10) by means of melting, in particular thermal melting, or using an adhesive, in particular a hot-melt adhesive, preferably by means of laminating.

24. The method as claimed in one of the preceding claims, wherein the capillary has a first aperture (16) and a second aperture (18), which apertures are preferably arranged at opposing ends of the capillary.

25. The method as claimed in claim 24, wherein the capillary is filled with liquid by way of the first aperture (16) and emptied by way of the first aperture (16) or the second aperture (18).

26. The method as claimed in one of the preceding claims, wherein the capillary is filled with the liquid and/or emptied using pressure or negative pressure.

27. The method as claimed in one of claims 24 to 26, wherein the first aperture (16) is arranged below the surface of the liquid and the second aperture (18) is arranged above the surface of the liquid.

28. The method as claimed in claims 24 to 27, wherein the first aperture (16) is sealed for implementing the reaction.

29. The method as claimed in one of claims 24 to 28, wherein the second aperture (18) has an internal width which is smaller than the internal width of the capillary.

30. The method as claimed in claim 29, wherein the internal width of the second aperture (18) is at most 0.3 mm, preferably at most 0.2 mm.

31. The method as claimed in one claims 24 to 30, wherein an appliance for the automated processing of samples is connected to the first aperture (16) and/or the second aperture (18), in particular by way of liquid-conducting channels which are contained in the device.

32. The method as claimed in one of the preceding claims, wherein the liquid is heated by heat being supplied to it through the plastic film (14) from a heating element (24) or by heat being supplied to it by a heating element (24) which is arranged within the capillary, within a wall of the capillary or within the plastic film (14).

33. The method as claimed in one of the preceding claims, wherein the liquid is heated or cooled by a gas stream or air stream of the appropriate temperature being blown onto the capillary.

34. The method as claimed in one of the preceding claims, wherein the capillary is only partly, in particular only to a maximum of 80% of its volume, filled with the liquid.

35. The method as claimed in claim 34, wherein heat is supplied to the capillary only in a region of the capillary which is filled with liquid such that the liquid which is vaporized in the reaction can condense in a residual region of the capillary, in particular a region which is arranged below the second aperture (18), and can flow back into the liquid which remains.

36. The method as claimed in claim 35, wherein the residual region of the capillary is cooled, in particular, by means of a Peltier element or an enlarged surface which radiates off heat.

37. The method as claimed in one of the preceding claims, wherein the method is used for the automated sample working-up, synthesis and/or analysis of biopolymers.

38. The method as claimed in one of the preceding claims, wherein the capillary is a component of a disposable unit which is, in particular, inserted into an appliance for the automated processing of samples.

39. The method as claimed in one of the preceding claims, wherein an outflow of heat from the capillary into parts of the substrate (10) is reduced by a means for interrupting the heat transfer, in particular a recess, which runs essentially parallel to the capillary, being arranged in the substrate (10), between the capillary and the parts of the substrate.

40. A device for carrying out a method as claimed in one of claims 1 to 39, containing at least one elongated reaction chamber (12) which has at least one first aperture (16), with at least one part of the longitudinal wall of the reaction chamber (12) being formed by a plastic film (14), wherein the reaction chamber (12) is formed as a capillary having a round or angular cross section in a substrate (10) by at least one elongated, channel-shaped recess which is entirely or partially sealed with the plastic film (14), wherein a means for interrupting the transfer of heat from the capillary into parts of the substrate (10) is provided in the substrate (10), between the capillary and the parts of the substrate (10).

41. The device as claimed in claim 40, wherein the substrate (10) consists of a plastic which is, in particular, hydrophobic and preferably rigid.

42. The device as claimed in claim 41, wherein the plastic is a plastic which does not inhibit a polymerase chain reaction (PCR), preferably polycarbonate, polypropylene or polyethylene.

43. The device as claimed in one of claims 40 to 42, wherein the substrate (10) is an injection-molded part.

44. The device as claimed in one of claims 40 to 43, wherein the recess in the substrate (10) is formed during the injection molding or, after that, by means of stamping, punching, milling or etching.

45. The device as claimed in one of claims 40 to 44, wherein the plastic film (14) is self-adhering.

46. The device as claimed in one of claims 40 to 45, wherein the plastic film (14) is bonded to the substrate (10) by means of melting, in particular thermal melting, or using an adhesive, in particular a hot-melt adhesive, preferably by means of laminating.

47. The device as claimed in one of claims 40 to 46, wherein the internal width of the capillary is at least 0.2 mm, preferably at least 0.3 mm.

48. The device as claimed in one of claims 40 to 47, wherein the internal width of the capillary is at most 3 mm, in particular at most 2mm.

49. The device as claimed in one of claims 40 to 48, wherein the cross sectional area of the capillary is maximally 10 mm², in particular maximally 4 mm², preferably maximally 2 mm².

50. The device as claimed in one of claims 40 to 49, wherein the length of the capillary is from 10 to 100 mm, in particular from 20 to 30 mm, and is preferably 25 mm.

51. The device as claimed in one of claims 40 to 50, wherein the plastic film (14) forms at least ⅕, preferably at least ¼, in particular at least ⅓, of the area of the longitudinal wall of the capillary.

52. The device as claimed in one of claims 40 to 51, wherein the volume of the reaction chamber (12) is from 0.5 to 500 μl, in particular from 5 to 50 μl, preferably from 10 to 30 μl.

53. The device as claimed in one of claims 40 to 52, wherein the reaction chamber (12) has a first aperture (16) and a second aperture (18).

54. The device as claimed in claim 53, wherein the first aperture (16) and the second aperture (18) are arranged at opposing ends of the reaction chamber (12).

55. The device as claimed in one of claims 40 to 54, wherein the first aperture (16) and/or the second aperture (18) is/are arranged in the substrate (10).

56. The device as claimed in one of claims 40 to 55, wherein the means for interrupting the transfer of heat from the capillary into parts of the substrate (10) is a recess which runs essentially parallel to the capillary.

57. The device as claimed in one of claims 40 to 56, wherein the first aperture (16) is arranged in a part of the reaction chamber (12) which is envisaged for taking up liquid and the second aperture (18) is arranged in the other part of the reaction chamber (12).

58. The device as claimed in one of claims 40 to 57, wherein the second aperture (18) has an internal width which is smaller than the internal width of the capillary.

59. The device as claimed in claim 58, wherein the internal width of the second aperture (18) is at most 0.3 mm, preferably at most 0.2 mm.

60. The device as claimed in one of claims 40 to 59, wherein the device contains a heating element (24), in particular within the reaction chamber (12), within a wall of the reaction chamber (12) or within the plastic film (14).

61. The device as claimed in claim 60, wherein the heating element (24) is arranged such that heat can only thereby be supplied to a region of the reaction chamber (12) which is envisaged for taking up liquid, so that vaporized liquid can condense in a residual region of the reaction chamber (12).

62. The device as claimed in claim 61, wherein a cooling element, in particular a Peltier element or an enlarged surface for radiating off heat, is provided at the residual region of the reaction chamber (12).

63. The device as claimed in one of claims 40 to 62, wherein the reaction chamber (12) is shaped such that, when the reaction chamber (12) is at a sufficient angle to the horizontal, gas bubbles which are formed in the liquid therein can ascend to the surface of the liquid without being retained, in this connection, at a projection, at a local high point or at a site in the reaction chamber (12) which does not have a sufficient angle to the horizontal.

64. The device as claimed in one of claims 40 to 63, wherein by when it is used as intended, the reaction chamber (12) has an angle to the horizontal of between 10° and 90°, in particular of between 45° and 90°, preferably of between 80° and 90°, preferably 90°.

65. The device as claimed in one of claims 40 to 64, wherein the reaction chamber (12) has a linear shape.

66. The device as claimed in one of claims 40 to 65, wherein a nucleic acid amplification reaction, in particular a polymerase chain reaction (PCR), is carried out in the method.

67. The device as claimed in one of claims 40 to 66, wherein the reaction chamber (12), the plastic film (14) and the adhesive, which is present, where appropriate, are selected such that they withstand a temperature of between 80° C. and 100° C., in particular of between 85° C. and 97° C., preferably 95° C.

68. The device as claimed in one of claims 40 to 67, wherein the reaction chamber (12) is connected, at the first and/or the second aperture (18), in particular by way of liquid-conducting channels which are contained in the device, to an appliance for the automated processing of samples, or can be connected to the appliance, in particular by insertion into the appliance.

69. The device as claimed in one of claims 40 to 68, wherein the device possesses several reaction chambers (12) which are, in particular, arranged parallel to each other.

70. The device as claimed in claim 69, wherein the plastic film (14) extends over several of the reaction chambers (12) and in each case forms a part of the longitudinal wall of each of these reaction chambers (12).

71. The device as claimed in one of claims 40 to 70, wherein reagents for carrying out the method, in particular reagents for implementing a PCR and/or a detergent, preferably in dry form, are contained in at least one of the reaction chambers (12).

72. The use of a detergent for changing at least one property of gas bubbles which are being formed in connection with a reaction in an unmoved liquid in a capillary which is open at at least one end, with the capillary having at least an angle to the horizontal which is such that the gas bubbles in the capillary ascend to a surface of the liquid.

73. The use as claimed in claim 72, wherein the detergent is a nonionic detergent, in particular Tween 20, Nonidet P40 or Triton X-100.

74. The use as claimed in claim 72 or 73, wherein the detergent is used at a concentration of from 0.01 to 5% (w/v), preferably of from 0.02 to 5% (w/v), most preferably of from 0.05 to 5% (w/v), in particular of from 0.1 to 5% (w/v) or of from 0.5 to 5% (w/v).

75. The use as claimed in one of claims 72 to 74, wherein the reaction is part of an automated sample working-up, synthesis and/or analysis of biopolymers.

76. The use as claimed in one of claims 72 to 75, wherein the capillary is a component of a disposable unit which is, in particular, inserted into an appliance for automated processing.

77. The use as claimed in one of claims 72 to 76, wherein the detergent is used in a method as claimed in one of claims 1 to 39.

78. The use as claimed in one of claims 72 to 77, wherein the capillary is provided by a device as claimed in one of claims 40 to 71.


SEQUENCE LISTING (6 sequences)

SEQ ID NO: 1 - DNA, 20 bp, Homo sapiens
gcctgaagaagtggatacag

SEQ ID NO: 2 - DNA, 19 bp, Homo sapiens
gaatagcactgggagcatt

SEQ ID NO: 3 - DNA, 21 bp, Homo sapiens
ggtttcgaagtggtggtcttg

SEQ ID NO: 4 - DNA, 20 bp, Homo sapiens
cctgccccaatccctttatt

SEQ ID NO: 5 - DNA, 22 bp, Homo sapiens
tgcccagtgcttaacaagacca

SEQ ID NO: 6 - DNA, 20 bp, Homo sapiens
tgttatcacactggtgctaa